(19) United States
(12) Patent Application Publication
(10) Pub. No.: US 2026/0286273 A1
(43) Pub. Date: Sep. 24, 2026

(54) BIOPROCESSING SYSTEM AND METHOD OF SEALING A DRIVE ASSEMBLY THEREOF

(71) Applicant: Global Life Sciences Solutions Operations UK Limited, Buckinghamshire (GB)

(72) Inventors: KEVIN ASHLEY LAMPORT, Hampshire (GB); IVAN JOHN REED, Hampshire (GB)

(73) Assignee: Global Life Sciences Solutions Operations UK Limited, Buckinghamshire (GB)

(21) Appl. No.: 19/084,864

(22) Filed: Mar. 20, 2025

(51) Int. Cl. C12M 1/12; C12M 1/00; C12M 1/06

(52) U.S. Cl. CPC C12M 37/04; C12M 23/26; C12M 27/02; C12M 37/06

(57) ABSTRACT

A drive assembly of a bioprocessing system is provided that includes a shaft controlled between an upper bearing assembly and a lower bearing assembly, a stationary seal portion configured to be stationary with respect to the shaft, and a rotary seal portion positioned above the stationary seal portion. The rotary seal portion is configured to rotate with the shaft. The stationary seal portion and the rotary seal portion are configured to inhibit microbial ingress into the bioprocessing system.

### BACKGROUND

### Technical Field

Examples of the subject matter herein relate generally to equipment used to manufacture chemical agents, particularly biopharmaceuticals, as well as sealing methods thereof. The equipment may include disposable containers.

### Discussion of Art

Bioprocessing systems used for manufacturing chemical and/or biological products have increasingly turned to single use containers or disposable containers.

Impellers are typically implemented in such containers and are rotated in order to mix the contents of the container. Such impellers can be mechanically driven, for example, through use of a drive shaft connected to a motor. Maintaining the reliability and sterility of these containers has been a challenge, especially when a drive shaft is used, because the shaft has to penetrate the bag from the external environment in order to be driven by an external motor.

Even small holes or leaks may allow contaminants (e.g., bacteria) to enter the system from the environment. Creating seals to join components of bioprocessing systems, specifically with an impeller drive shaft and the container in mind, may be an especially difficult challenge with regard to maintaining sterility, reliability, and longevity. In many bioprocessing applications, the primary fluids are aqueous in nature. As such, the primary fluids have little innate lubricity, which makes it difficult to use simple lip seals for extended durations in dynamic applications.

Because of the increasingly disposable nature of containers in bioprocessing systems, mechanical seals generally used in other contexts, for example stainless steel processing, are considered to be overkill for a disposable solution intended to be used for a relatively short period of time and then disposed of. Moreover, such mechanical seals can generate particulates, in the context of an impeller shaft, which are undesirable in a sterile setting. As such, mechanical seals are not typically used in bioprocessing systems using disposable or single use containers.

It is possible to avoid use of seals altogether, for example, by use of a magnetic coupling between the impeller and the motor. But such configurations are expensive and often do not provide enough torque to adequately mix larger volumes of fluid.

There is thus a need for an improved design and methods of use of sealing techniques in bioprocessing systems in order to facilitate a more reliable, sterile, efficient and intensive processes.

### BRIEF DESCRIPTION

In accordance with an aspect of the invention, a drive assembly of a bioprocessing system is described. The drive assembly includes a shaft, a stationary seal portion, and a rotary seal portion. The shaft is controlled between an upper bearing assembly and a lower bearing assembly. The stationary seal portion is configured to be stationary with respect to the shaft. The rotary seal portion is positioned above the stationary seal portion. The rotary seal portion is configured to rotate with the shaft. The stationary seal portion and the rotary seal portion are configured to inhibit microbial ingress into the bioprocessing system.

Mechanical seals may operate on three mechanisms of fluid exclusion: centrifugal throw, compressive squeeze, and vaporization at the inner portion of the seal contact faces while maintaining a thin film of fluid around a single micron in thickness between the two dynamic faces. The faster these seals operate, the better their mechanical sealing attributes perform and thus it has been an improvement in the drive development to select materials of constructions between these two faces that have compatible hardness, lubricity, and wear profiles to support predetermined speeds of operation.

In one embodiment, the rotary seal portion may be configured to provide bidirectional rotation. The shaft may be connected to an impeller located within a sterile bioprocessing container. The sterile bioprocessing container may be a flexible container. The rotary seal portion may include silicone carbide and the stationary seal portion may be carbon graphite. In another embodiment, the stationary seal portion may include silicone carbide and the rotary seal portion may be carbon graphite.

In one example, the drive assembly may include a leak path. The leak path may be configured to test a sealing efficiency of the drive assembly as a post manufacture and/or post use integrity assurance step.

In accordance with another aspect of the invention, a drive assembly of a bioprocessing system is described. The drive assembly includes a shaft, a static seal face, and a rotary seal face. The shaft is controlled between an upper bearing assembly and a lower bearing assembly. The static seal face may be configured to be stationary with respect to the shaft. The rotary seal face may be positioned above the static seal face. The rotary seal face may be configured to rotate with the shaft. The static seal face and the rotary seal face may be configured to inhibit microbial ingress into the bioprocessing system.

In an embodiment, the drive assembly may be configured to include a smooth body having hygienic properties.

In one embodiment, the shaft may be connected to an impeller located within a sterile bioprocessing container. The sterile bioprocessing container may be flexible.

In one embodiment, the drive assembly may include one or more springs that may be configured to provide a pre-load force to the rotary seal face, such that the rotary seal face is positioned at a predetermined working height.

In one embodiment, the drive assembly may include a leak path. The leak path may be configured to test a sealing efficiency of the drive assembly.

In one embodiment, the rotary seal may be coupled to the shaft by friction fit. The rotary seal face may include silicone carbide and the static seal face may be carbon graphite.

In accordance with yet another aspect of the invention, a method of sealing a drive assembly of a bioprocessing system is provided. The method may include providing a shaft of the drive assembly between an upper bearing assembly and a lower bearing assembly. The method may include providing a stationary seal portion about the shaft. The stationary seal portion may be configured to be stationary with respect to the shaft. The method may include positioning a rotary seal portion above the stationary seal portion. The rotary seal portion may be configured to rotate with the shaft. The method may include inhibiting microbial ingress into the drive assembly using the stationary seal portion and the rotary seal portion.

In an embodiment, the method may include providing a tortious path adjacent the stationary seal portion to prevent egress of fluid from the drive assembly.

In one embodiment the method may include providing a leak path that may be configured to test a sealing efficiency of the drive assembly.

In one embodiment, one of the stationary seal portion or the rotary seal portion include silicone carbide and the other of the stationary seal portion or the rotary seal portion is carbon graphite.

In one embodiment, the stationary seal portion and the rotary seal portion are configured to prevent microbial ingress into the bioprocessing system.

In one embodiment, the drive assembly may be configured to include a smooth body having a hygienic properties.

In one embodiment, the shaft may be connected to an impeller located within a sterile bioprocessing container. The sterile bioprocessing container may be flexible.

In one example, the method further may include one or more springs providing a pre-load force to the rotary seal portion, such that the rotary seal portion may be positioned at a predetermined working height.

In one embodiment the rotational translation from the shaft to the rotary portion of the seal can be coupled by mechanical means.

### BRIEF DESCRIPTION OF THE DRAWINGS

The subject matter may be understood from reading the following description of non-limiting examples, with reference to the attached drawings, wherein below:

FIG. 1 is a cross-sectional view of a bioprocessing system, according to an embodiment of the invention;

FIG. 2 is a cross-sectional view of a drive assembly for a bioprocessing system, according to an embodiment of the invention;

FIG. 3 is a cross-sectional view of a drive assembly for a bioprocessing system, according to an embodiment of the invention;

FIG. 4 is a cross-sectional view of a drive assembly for a bioprocessing system, according to an embodiment of the invention;

FIG. 5a is a cross-sectional view of a portion of a drive assembly for a bioprocessing system, according to an embodiment of the invention; and

FIG. 5b is a cross-sectional view of a drive assembly for a bioprocessing system, according to an embodiment of the invention.

### DETAILED DESCRIPTION

Unless defined otherwise, all technical and scientific terms used herein have the same meaning as commonly understood by one of ordinary skill in the art to which this invention pertains.

Embodiments of the subject matter described herein may relate to a bioprocessing system and a drive assembly thereof. The drive assembly may be coupled with a motor to drive an impeller. One aim of the present subject matter is to maintain the reliability and sterility of the system. When a drive shaft is used, the shaft has to penetrate a bag of the system from the external environment in order to be driven by an external motor. A sealing system and method is describe that is configured to inhibit microbial ingress into the bioprocessing system, and thus to maintain the reliability and sterility of the system.

The presently described bioprocessing systems may be used for the manufacture of chemical and/or biological products, for example, biopharmaceuticals. The bioprocessing system may utilize disposable containers. The use of single use containers or disposable containers in manufacturing processes has increased significantly in recent years. Maintaining the sterility during manufacture and use remains a challenge. Sterile and fluid tight seals between components present a particularly difficult challenge as the chances of contaminating in such areas are high. As such, improved sealing efficacy is of utmost importance.

In embodiments of the present disclosure, the sealing technique may include an energized mechanical seal with silicone carbide/carbon graphite sealing faces. This sealing technique is beneficial, even in single use bioprocessing systems, for reasons discussed further below. Bioprocessing systems are operating at increased intensity and may require continuous processing needs. As such, improved sealing techniques are beneficial. The sealing techniques discussed herein provide integrity assurance, reliability, and increased longevity. These sealing techniques provide peace of mind to the users on system reliability in high stress environments.

In one embodiment, the bioprocessing system includes a drive having a shaft controlled between two bearings to maintain control of the overall system. Above an upper bearing may be a seal carrier which allows housing of the lower sealing seat of a seal assembly. The sealing portion may remain static in relation to the drive housing whilst the upper portion of the seal which houses the upper sealing face and a sprung energized portion of the seal is affixed to the shaft by friction between upper O-rings in the upper assembly.

A housing having a rotary shaft seal setup inherently has the ability to leak into the system. For example, a rotary shaft seal setup may include a theoretical leak path through the dynamic portion of the seal arrangement. During operation of the bioprocessing system, two of the major areas of concern are: 1. breach of containment of the process fluid to the operational environment such that it could contaminate the environment or pose health risk to the human operators in that environment, and 2. ingress of contaminant, commonly in the form of bacteria or virus, that could cause the batch of cell growth to be contaminated and thus scrapped.

These concerns may be tested and validated through a wide range of characteristics and results. These tests may be run on the below described subject matter to ensure and validate that the described system addresses the major areas of concern discussed. The tests may be pass/fail criteria or may have certain predetermined limits. The tests may include whether any process fluid is observed on any external surface of the drive housing, lower bearing, or the drive coupling/shaft. Process fluid, in this context, may include a gross leak and a significant risk of batch contamination, if not already occurred.

In another example, the bioprocessing system and components thereof may include a test including a determinization of whether or not there has been an accumulation of fluid mass inside the drive that is significant enough to overcome its own surface tension and run down the drive to form and accumulated mass inside the drive that could form a transient path for contaminating microorganisms to move from the lower portion of the drive at the lower bearing to the dynamic seal face.

In another example, the bioprocessing system and components thereof may be tested to determine whether the drives will pass an intensive run of bacterial challenge tests fun over the full life profile of the systems expected operational life.

There are several reasons or factors that may result in fluid being noted inside of the drive housing. The reasons may include but not be limited to condensation, speed of operation, system pressure, seal wear or damage during transit and assembly.

Mechanical seals may operate on three mechanisms of fluid exclusion: centrifugal throw, compressive squeeze, and vaporization at the inner portion of the seal contact faces while maintaining a thin film of fluid, around a single micron in thickness, between the two dynamic faces. The faster these seals operate, the better their mechanical sealing attributes perform and thus it has been an improvement in the drive development to select materials of constructions between these two faces that have compatible hardness, lubricity, and wear profiles to support predetermined speeds of operation.

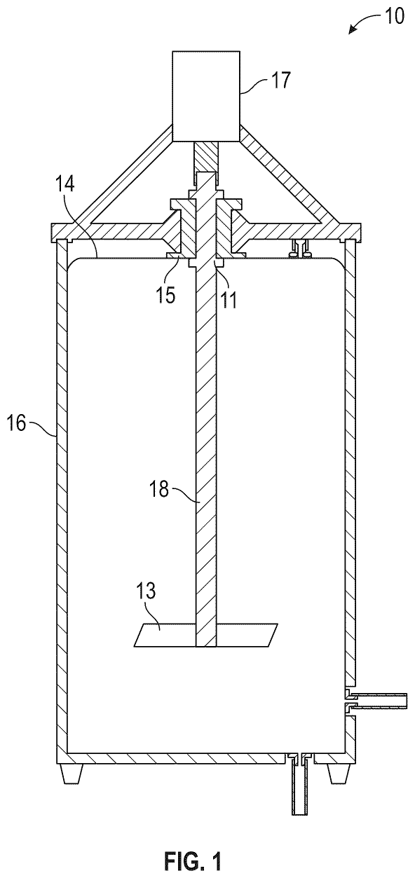

FIG. 1 illustrates a bioprocessing system 10, according to one embodiment. The bioprocessing system may include single use bioreactors, stirred tank reactors, or the like. The bioprocessing system 10 may include a housing 16 and a bag 14, for example a flexible bag. The bioprocessing system 10 may include an agitation shaft 18 with an impeller 13, which may be positioned inside of the bag 14. The impeller 13 may be rotated in order to mix the contents of the bag 14. The impeller 13 may be driven, for example, through use of the shaft 18 connected to a motor 17.

The shaft 18 may be coupled with a bearing 15 and may include one or more seals 11. The motor 17 may be positioned to drive the shaft 18 and configured to drive the impeller 13 and facilitate mixing in the bioprocessing system. In one example, the bioprocessing system, and the components thereof, may be disposable. The bioprocessing system 10 illustrated in FIG. 1 shows a top driven system, i.e., where the motor is at the top of the system. However, in other embodiments, the bioprocessing system may be a bottom driven system with a motor at the bottom of the system or a laterally driven system with a motor at a lateral portion of the system.

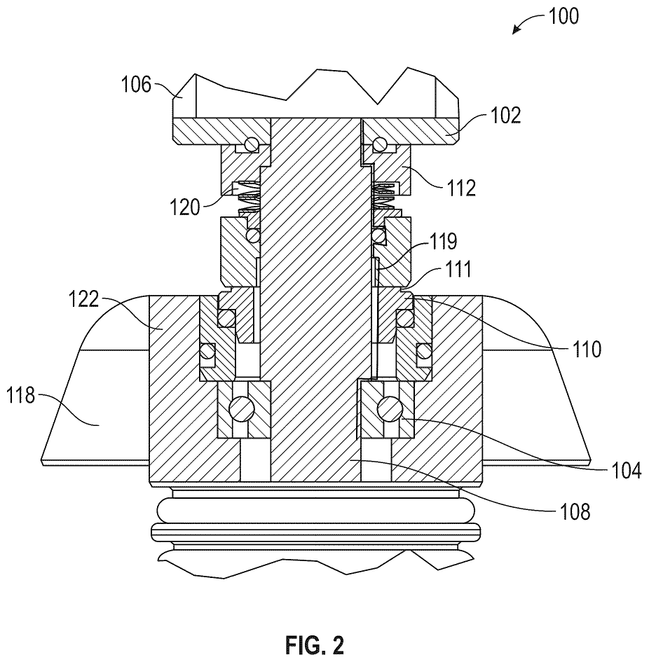

FIG. 2 illustrates a drive assembly 100 of a bioprocessing system, according to one embodiment. The bioprocessing system may include single use components. The drive assembly 100 may be a direct drive assembly. In other embodiments, the drive assembly may be an alternate drive assembly. The direct drive assembly 100 may be able to provide greater levels of power, compared to alternate drive systems. A traditional disadvantage of direct drive systems is that the direct drive systems are prone to leakage because seals may generate leak paths during use. For example, the direct drive systems for driving an impeller in a bioprocessing system may require a lot of rotations during use that increase the probability of leakage. Additionally, traditional direct drive systems may have a theoretical leak path to the external environment which needs to both exclude microbial ingress to prevent batch contamination and maintain a wear profile on the selected seals such that seal performance can be sustained over the intended run life to ensure system integrity. The present drive assembly is designed to address this traditional disadvantage by implementing a mechanical seal.

In one example, the mechanical seal arrangement may be used to ensure adequate sealing performance and increased longevity of the bioprocessing system. The sealing technique may include an energized mechanical seal with silicone carbide/carbon graphite sealing faces. This sealing technique accommodates the increased intensity and continuous processing needs presently in use, while offering a high level of integrity assurance for extended durations. This may provide customers peace of mind on system reliability in high stress environments.

The drive assembly 100 includes a shaft 108 connected to impeller 106 (partially shown) that may be controlled between an upper bearing assembly 102 and a lower bearing assembly 104. A stationary seal portion 110 is positioned around the shaft 108 and is configured to be stationary with respect to the shaft 108. The stationary seal portion 110 remains static in relation to drive assembly 100 and may be positioned within a seal housing 118.

A rotary seal portion 112 may be positioned above the stationary seal portion 110 and may be configured to rotate about the shaft 108. The rotary seal portion 112 may be rigidly attached to the shaft and may rotate relative to the stationary seal portion 110 and with the shaft 108. The stationary seal portion 110 and the rotary seal portion 112 are configured to inhibit ingress into the bioprocessing system (e.g., microbial ingress). The rotary seal portion 112 may be affixed to the shaft 108 by friction fit. One or more O-rings 122 may be positioned adjacent to components to enhance the friction fit. A spring energized portion 120 may be affixed to the shaft 108, adjacent to the rotary seal portion 112. The spring energized portion 120 may include one or more springs. The spring energized portion 120 may apply a pre-load force on the rotary seal portion 112 to set a working height of an impeller adapter. The pre-load force may determine or govern the pressure on the sealing faces of the rotary and stationary seal portions 110, 112 from the springs in an upper portion of the sealing arrangement. In one example, the rotary seal portion 112 may provide bidirectional rotation. The bidirectionality of the rotary seal portion 112 is useful in the current landscape to meet wide ranging preferences and uses by end users of the bioprocessing system.

While in one embodiment, the rotary seal portion 112 may be above the stationary seal portion 110 in a vertical direction, in other embodiments, the seal portions may have different orientations. For example, the rotary seal portion 112 may be below the stationary seal portion 110 in the vertical direction. The seals may be oriented horizontally, and the rotary seal portion 112 may be positioned to the left or the right of the stationary seal portion 110. Additionally, the rotary seal portion 112 may be positioned externally to the stationary seal portion 110. In another embodiment, the rotary seal portion 112 may be positioned internally to the stationary seal portion 110. The arrangement of the rotary seal portion 112 relative to the stationary seal portion 110 may be selected based on desired use characteristics and function. The rotary seal portion 112 and the stationary seal portion 110 may be oriented such that the process fluid is in contact with an external diameter of the seal and the sealing faces are occluding fluid from moving to an internal cavity of the drive assembly.

In one embodiment, one or both of the stationary seal portion 110 and the rotary seal portion 112 may include a tortuous path 119 to inhibit egress of fluid from the drive assembly. The tortuous path is defined by the contours and shapes of the components, specifically the bearing assemblies 102 and 104, the stationary seal portion 110, and the rotary seal portion 112. The seal portions are shaped with various protrusions and contours that prevent a direct flow of fluid, thus enhancing the tortuous path. The tortuous path may be a sealed torturous path up to the primary fluid seal, which is created by the lower bearing in the drive assembly which may be a press fitted bearing. The sealed torturous path may be defined as the path extending between the lower bearing assembly 104 and the upper bearing assembly 102. The primary fluid seal may be formed by the lower bearing assembly 104. The vertical orientation of the drive assembly at the base of the system, in combination with the span between the lower bearing and the primary fluid seal means that any microbe would have to have some motive force to get towards the primary fluid seal. As such, this sealing technique significantly reduces microbial ingress risk.

Another aspect of the present invention relates to the materials used for the drive assembly. In bioprocessing applications, the primary fluids are aqueous in nature and, as such, have minimal innate lubricity. These characteristics makes it extremely difficult to employ simple lip seals for extended run durations. However, the present sealing arrangement is not affected by the lack of lubricity in aqueous media. Additionally, the present sealing arrangement produces less particulate accumulation and shedding than similar lip sealing arrangements and. In one embodiment, the stationary seal portion 110 includes a ceramic seal face 111, for example silicone carbide, and the rotary seal portion 112 is carbon graphite. In one example, the rotary seal portion 112 may include a ceramic seal face. In yet another example, both the stationary seal portion 110 and the rotary seal portion 112 may include a ceramic seal face.

In another embodiment, the stationary seal portion 110 may be carbon graphite and the rotary seal portion 112 may include a ceramic material. The ceramic material is a relatively hard and polished material compared to the softer carbon graphite portion. As such, the interaction between the materials may wear the carbon graphite to a matched and polished condition. The interaction produces small amounts of shed. The two materials match and accommodate the contours of the other to improve sealing of the overall system. In other embodiments, the stationary seal portion and the rotary seal portion may be made of the same material, for example, both carbon graphite or both ceramic or a combination of the two.

An additional benefit of the carbon graphite sealing portion is that the sealing face may hover on a small amount of process fluid, for example a micron film thickness. This creates virtually zero contact, which results in very low wear because of the limited mechanical interaction. As such, the longevity is significantly increased.

In one embodiment, an upper portion of the drive assembly 100, for example the upper bearing 102, may have a smooth body. The upper portion may include stainless steel. The smooth body may increase the hygienic properties as there are fewer contours and areas for contaminants to accumulate. This design may also reduce or eliminate the need for intensive cleaning using, for example, caustic fluids or steam, due to the hygienic properties of the smooth body. This design not only reduces contamination, but also reduces the time and effort to maintain and/or clean the drive assembly 100 prior to assembly into a product.

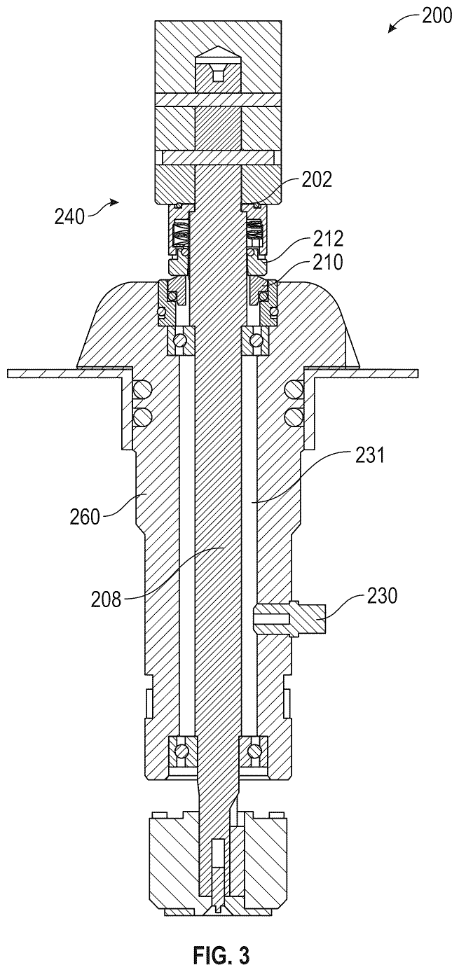

FIG. 3 illustrates a cross-sectional view of a drive assembly 200, according to one embodiment. The drive assembly 200 includes a shaft 208 of an impeller that may be controlled between an upper bearing assembly and a lower bearing assembly. The shaft 208 may be surrounded by a housing 260 of the drive assembly 200. A stationary seal portion 210 may be positioned around the shaft 208 and is configured to be stationary with respect to the shaft 208. A rotary seal portion 212 may be positioned above the stationary seal portion 210 and may be configured to rotate about the shaft 208. The rotary seal portion 212 may be rigidly attached to the shaft 208 and may rotate relative to the stationary seal portion 210 and with the shaft 208.

As illustrated in FIG. 3, the drive assembly 200 may also include a leak path 231 in fluid communication with a bleed hole 230 that is positioned and designed to test a sealing efficiency of the drive assembly. This leak path is designed as a quality assurance feature of the drive assembly. The intentional leak may be introduced to bypass a bearing 202. This introduction ensures that the seals 210, 212 are inhibiting the leakage, rather than the bearing 202. A process fluid side 240 may be charged with pressure to move the fluid through the system. If any leakage is detected at the bleed hole 230, this may indicate a sealing issue. A measurement may be taken of the leak through the system and out the bleed hole 230 that is in fluid communication with the leak path. This measurement may confirm whether the seals are functioning as expected. Once the seals are confirmed to be in functioning operations, the bleed hole 230 is plugged and the drive assembly is ready for use.

A common method for leak detection is the pressure decay method. In this method, the assembly is first filled with gas to a predetermined pressure. The gas is then left to stabilize for a predetermined amount of timed, and then the pressure is re-measured. If the pressure has decreased, this indicates that some of the gas has escaped from the assembly, and the precise drop in pressure can be correlated to the size of the defect, for example one or more holes. However, using the intentional leak path, described above, may improve the accuracy and efficiency of the leak detection un-occluded by the upper bearing.

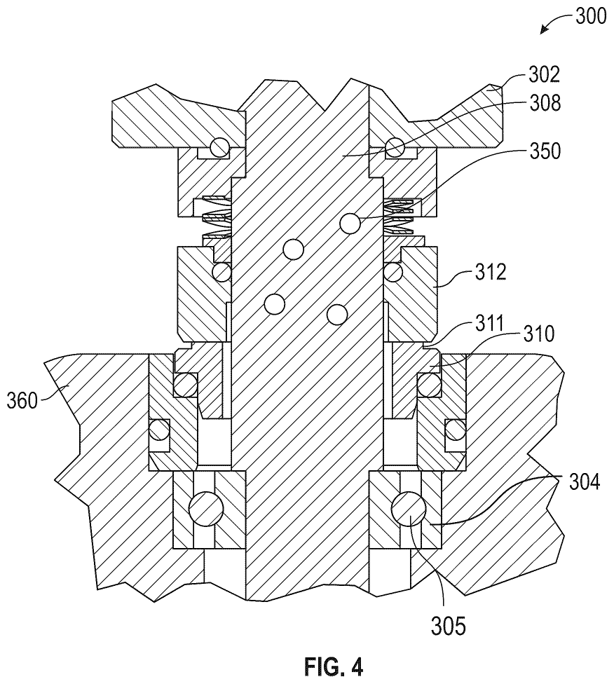

FIG. 4 illustrates a cross-sectional view of a portion of a drive assembly 300 illustrated in FIG. 3, according to one embodiment. The drive assembly 300 includes a shaft 308 of an impeller that may be controlled between an upper bearing assembly 302 and a lower bearing assembly 304. The shaft 308 may be surrounded by a housing 360 of the drive assembly 300. A stationary seal portion 310 may be positioned around the shaft 308 and is configured to be stationary with respect to the shaft 308. A rotary seal portion 312 may be positioned above the stationary seal portion 310 and may be configured to rotate about the shaft 308. The rotary seal portion 312 may be rigidly attached to the shaft and may rotate relative to the stationary seal portion 310 and with the shaft 308.

Identification of fluid internally within the drive below the upper bearing 302 or externally on any of the drive surfaces below the lower O-ring seal 305 of the bag port would constitute non-permissible bypass. As shown in FIG. 4, permissible droplets 350 are shown that generally will not conglomerate into a singular larger mass of fluid.

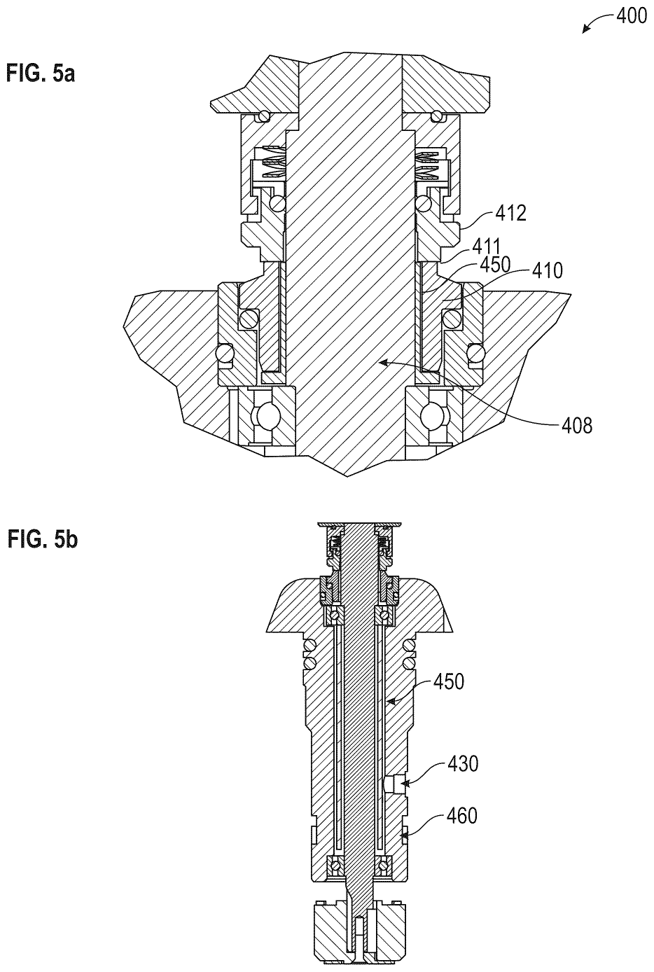

FIG. 5a illustrates a cross-sectional view of a portion of a drive assembly 400, according to one embodiment. FIG. 5b illustrates a cross-sectional view of the drive assembly 400, according to one embodiment. The drive assembly 400 includes a shaft 408 of an impeller that may be controlled between an upper bearing assembly and a lower bearing assembly. The shaft 408 may be surrounded by a housing 460 of the drive assembly 400. A stationary seal portion 410 may be positioned around the shaft 408 and is configured to be stationary with respect to the shaft 408. A rotary seal portion 412 may be positioned above the stationary seal portion 410 and may be configured to rotate about the shaft 408. The rotary seal portion 412 may be rigidly attached to the shaft and may rotate relative to the stationary seal portion 410 and with the shaft 408. Fluid accumulated in the area indicated 450 may illustrate impermissible fluid accumulation, according to one example. Aggregation in a cavity between a shaft 408 and a housing 460 may be impermissible fluid accumulation, according to one example.

The drive assembly 400 may include a seal face 411 positioned between the stationary seal portion 410 and the rotary seal portion 412. The seal face 411 is positioned where there is an interface between the two seal portions 410 and 412. In one embodiment, the seal face 411 may be a ceramic seal face, for example a silicone carbide seal face.

In one embodiment, a drive assembly of a bioprocessing system is described. The drive assembly includes a shaft, a stationary seal portion, and a rotary seal portion. The shaft is controlled between an upper bearing assembly and a lower bearing assembly. The stationary seal portion is configured to be stationary with respect to the shaft. The rotary seal portion is positioned above the stationary seal portion. The rotary seal portion is configured to rotate with the shaft. The stationary seal portion and the rotary seal portion are configured to inhibit microbial ingress into the bioprocessing system.

There are several reasons or factors that may result in fluid being noted inside of the drive housing. The reasons may include but not be limited to condensation, speed of operation, system pressure, seal wear or damage during transit and assembly.

In one example, the rotary seal portion may be configured to include a tortuous path to inhibit egress of fluid from the drive assembly. The stationary seal portion may be configured to include a tortuous path to inhibit egress of fluid from the drive assembly.

In one example, the rotary seal portion may be configured to provide bidirectional rotation. The shaft may be connected to an impeller located within a sterile bioprocessing container. The sterile bioprocessing container may be flexible. The rotary seal portion may include silicone carbide and the stationary seal portion may be carbon graphite. In another example, the stationary seal portion may include silicone carbide and the rotary seal portion may be carbon graphite.

In one example, the drive assembly may include a leak path. The leak path may be configured to test a sealing efficiency of the drive assembly.

In one embodiment, a drive assembly of a bioprocessing system is described. The drive assembly includes a shaft, a static seal face, and a rotary seal face. The shaft is controlled between an upper bearing assembly and a lower bearing assembly. The static seal face may be configured to be stationary with respect to the shaft. The rotary seal face may be positioned above the static seal face. The rotary seal face may be configured to rotate with the shaft. The static seal face and the rotary seal face may be configured to inhibit microbial ingress into the bioprocessing system.

In one example, the drive assembly may be configured to include a smooth body having hygienic properties. The shaft may be connected to an impeller located within a sterile bioprocessing container. The sterile bioprocessing container may be flexible.

In one example, the drive assembly may include one or more springs that may be configured to provide a pre-load force to the rotary seal face, such that the rotary seal face is positioned at a predetermined working height.

In one example, the drive assembly may include a leak path. The leak path may be configured to test a sealing efficiency of the drive assembly.

In one example, the rotary seal may be coupled to the shaft by friction fit. The rotary seal face may include silicone carbide and the static seal face may be carbon graphite.

In one embodiment, a method of sealing a drive assembly of a bioprocessing system is provided. The method may include providing a shaft of the drive assembly between an upper bearing assembly and a lower bearing assembly. The method may include providing a stationary seal portion about the shaft. The stationary seal portion may be configured to be stationary with respect to the shaft. The method may include positioning a rotary seal portion above the stationary seal portion. The rotary seal portion may be configured to rotate with the shaft. The method may include inhibiting microbial ingress into the drive assembly using the stationary seal portion and the rotary seal portion.

In one example, the method may include providing a tortious path adjacent the stationary seal portion to prevent egress of fluid from the drive assembly.

In one example, the method may include providing a leak path that may be configured to test a sealing efficiency of the drive assembly.

In one example, one of the stationary seal portion or the rotary seal portion include silicone carbide and the other of the stationary seal portion or the rotary seal portion is carbon graphite.

In one example, the stationary seal portion and the rotary seal portion are configured to prevent microbial ingress into the bioprocessing system.

In one example, the drive assembly may be configured to include a smooth body having a hygienic properties. In one example, the shaft may be connected to an impeller located within a sterile bioprocessing container. The sterile bioprocessing container may be flexible.

In one example, the method further may include one or more springs providing a pre-load force to the rotary seal portion, such that the rotary seal portion may be positioned at a predetermined working height.

As used herein, an element or step recited in the singular and proceeded with the word “a” or “an” do not exclude the plural of said elements or operations, unless such exclusion is explicitly stated. Furthermore, references to “one embodiment” of the invention do not exclude the existence of additional embodiments that incorporate the recited features. Moreover, unless explicitly stated to the contrary, embodiments “comprising,” “comprises,” “including,” “includes,” “having,” or “has” an element or a plurality of elements having a particular property may include additional such elements not having that property. In the appended claims, the terms “including” and “in which” are used as the plain-English equivalents of the respective terms “comprising” and “wherein.” Moreover, in the following claims, the terms “first,” “second,” and “third,” etc. are used merely as labels, and do not impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function devoid of further structure.

The above description is illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein define the parameters of the inventive subject matter, they are exemplary embodiments. Other embodiments will be apparent to one of ordinary skill in the art upon reviewing the above description. The scope of the inventive subject matter should, therefore, be determined with reference to the appended claims, along with the full scope of equivalents to which such claims are entitled.

Use of phrases such as “one or more of . . . and,” “one or more of . . . or,” “at least one of . . . and,” and “at least one of . . . or” are meant to encompass including only a single one of the items used in connection with the phrase, at least one of each one of the items used in connection with the phrase, or multiple ones of any or each of the items used in connection with the phrase. For example, “one or more of A, B, and C,” “one or more of A, B, or C,” “at least one of A, B, and C,” and “at least one of A, B, or C” each can mean (1) at least one A, (2) at least one B, (3) at least one C, (4) at least one A and at least one B, (5) at least one A, at least one B, and at least one C, (6) at least one B and at least one C, or (7) at least one A and at least one C.

This written description uses examples to disclose several embodiments of the inventive subject matter, including the best mode, and to enable one of ordinary skill in the art to practice the embodiments of inventive subject matter, including making and using any devices or systems and performing any incorporated methods. The patentable scope of the inventive subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A drive assembly of a bioprocessing system, comprising:
a shaft controlled between an upper bearing assembly and a lower bearing assembly;
a stationary seal portion configured to be stationary with respect to the shaft;
a rotary seal portion positioned above the stationary seal portion, the rotary seal portion configured to rotate with the shaft;
wherein the stationary seal portion and the rotary seal portion are configured to inhibit microbial ingress into the bioprocessing system.

2. The drive assembly of claim 1, further comprising one or more springs configured to provide a pre-load force to the rotary seal portion, such that the rotary seal portion is positioned at a predetermined working height.

3. The drive assembly of claim 1, wherein the stationary seal portion is configured to include a tortuous path to inhibit ingress/egress of media through the drive assembly.

4. The drive assembly of claim 1, wherein the shaft is connected to an impeller located within a sterile bioprocessing container, wherein the sterile bioprocessing container is flexible.

5. The drive assembly of claim 1, wherein the rotary seal portion includes silicone carbide and the stationary seal portion is carbon graphite.

6. The drive assembly of claim 1, wherein the stationary seal portion includes silicone carbide and the rotary seal portion is carbon graphite.

7. The drive assembly of claim 1, further comprising a leak path, wherein the leak path is configured to test a sealing efficiency of the drive assembly.

8. A drive assembly of a bioprocessing system, comprising:
a shaft controlled between an upper bearing assembly and a lower bearing assembly;
a static seal face configured to be stationary with respect to the shaft;
a rotary seal face positioned above the static seal face, the rotary seal face configured to rotate with the shaft;
wherein the static seal face and the rotary seal face are configured to inhibit microbial ingress into the bioprocessing system.

9. The drive assembly of claim 8, wherein the shaft is connected to an impeller located within a sterile bioprocessing container.

10. The drive assembly of claim 8, further comprising one or more springs configured to provide a pre-load force to the rotary seal face, such that the rotary seal face is positioned at a predetermined working height.

11. The drive assembly of claim 8, further comprising a leak path, wherein the leak path is configured to test a sealing efficiency of the drive assembly.

12. The drive assembly of claim 8, wherein rotary seal face is coupled to the shaft by friction fit.

13. The drive assembly of claim 8, wherein the rotary seal face includes silicone carbide and the static seal face is carbon graphite.

14. A method of sealing a drive assembly of a bioprocessing system, comprising:
providing a shaft of the drive assembly between an upper bearing assembly and a lower bearing assembly;
providing a stationary seal portion about the shaft, wherein the stationary seal portion is configured to be stationary with respect to the shaft;
positioning a rotary seal portion above the stationary seal portion, wherein the rotary seal portion is configured to rotate with the shaft; and
inhibiting microbial ingress into the drive assembly using the stationary seal portion and the rotary seal portion.

15. The method according to claim 14, further comprising providing a tortuous path adjacent the stationary seal portion to prevent egress of fluid from the drive assembly.

16. The method according to claim 14, further comprising providing a leak path configured to test a sealing efficiency of the drive assembly.

17. The method according to claim 14, wherein one of the stationary seal portion or the rotary seal portion include silicone carbide and the other of the stationary seal portion or the rotary seal portion is carbon graphite.

18. The method according to claim 14, wherein the stationary seal portion and the rotary seal portion are configured to prevent microbial ingress into the bioprocessing system.

19. The method according to claim 14wherein the shaft is connected to an impeller located within a sterile bioprocessing container.

20. The method according to claim 14, further comprising one or more springs providing a pre-load force to the rotary seal portion, such that the rotary seal portion is positioned at a predetermined working height.
